(19) United States
(12) Patent Application Publication
(10) Pub. No.: US 2004/0073241 A1
(43) Pub. Date: Apr. 15, 2004

(54) Implantable tissue constriction device and method for suppressing leakage of fluid from resectioned body tissue

(75) Inventors: Robert Lawrence Barry, Kirkland, WA (US); Clint Finger, Bellevue, WA (US)

Correspondence Address: KNOBBE MARTENS OLSON & BEAR LLP, 2040 MAIN STREET, FOURTEENTH FLOOR, IRVINE, CA, 92614, US

(73) Assignee: Spiration, Inc.

(21) Appl. No.: 10/270,036

(22) Filed: Oct. 11, 2002

(51) Int. Cl. A61B 17/08

(52) U.S. Cl. 606/157

(57) ABSTRACT

The invention provides an implantable tissue constriction device and method for suppressing leakage from resectioned body tissue. The device includes a first elongated member having a constricting surface, and a second elongated member having another constricting surface, the constricting surfaces being arranged in combination to circumscribe tissue to be excised, and further being moveable together in opposition to constrict tissue adjacent to the circumscribed tissue to be excised to an extent necessary to suppress leakage from body tissue remaining after excision of the tissue to be excised. One elongated constrictive surface may include a pliable material arranged to contact the circumscribed tissue and accommodate anatomical variations. The device may include a pivot coupled between the first and second elongated members, about which the constricting surfaces are moveable together in opposition. The device may include a bias element coupled between the first and second member that brings the constricting surfaces together in opposition.

### BACKGROUND

The present invention is generally directed to a device and method for constrictively suppressing leaks from resectioned body tissue. The present invention is more particularly directed to an implantable device for constrictively isolating tissue to be excised, and suppressing blood and air leaks from resectioned body tissue after excision of the tissue to be excised.

A number of open and minimally invasive surgical procedures involve resecting tissue and organs that are prone to short- and long-term leakage of blood and other fluids. In addition, injuries may cause leakage that need to be repaired. Many organs are difficult to seal against leakage because of their structure and the type of fluids present. Some organs involve particularly small or difficult tissue to suture or cauterize, making resectioning those organs particularly difficult and fraught with complications. For example, lung tissue includes thin, fragile, and slippery blood vessels and air passageways that are difficult to suture against leaks. After the diseased tissue is removed, the remaining or resectioned lung portion is often restructured with suture staples. In about thirty percent of these cases, sutured lung tissue leaks air because the vessels were not adequately sealed. In other cases, sutured lung tissue leaks blood from the resection site for the same reason. Treatment for such leaks depends upon their severity, and often requires further open-chest surgery.

In view of the foregoing, there is a need in the art for a new and improved minimally invasive implantable device and method for suppressing leaks from resectioned body tissue without suturing. The present invention provides an implantable tissue constriction device and method for suppressing leaks in organs and tissue without suturing.

### SUMMARY

The invention provides an implantable tissue constriction device for suppressing leakage from body tissue. The device includes a first elongated member having a constricting surface, and a second elongated member having another constricting surface, the constricting surfaces being arranged in combination to circumscribe a portion of the body tissue, and further being moveable together in opposition to constrict body tissue adjacent to the circumscribed body tissue portion to an extent necessary to suppress leakage from the body tissue.

The invention also provides an implantable tissue constriction device for suppressing leakage from resectioned body tissue. The device includes a first elongated member having a constricting surface, and a second elongated member having another constricting surface, the constricting surfaces being arranged in combination to circumscribe tissue to be excised, and further being moveable together in opposition to constrict tissue adjacent to the circumscribed tissue to be excised to an extent necessary to suppress leakage from body tissue remaining after excision of the tissue to be excised. At least one elongated constrictive surface may include a pliable material arranged to contact the circumscribed tissue and accommodate anatomical variations. The device may include a pivot coupled between the first elongated member and the second elongated member, about which the constricting surfaces are moveable together in opposition. The device may include a first pivot and a second pivot, each pivot coupling the first constrictive surface to the second constrictive surface, and cooperatively allowing movement of the constricting surfaces together in opposition. At least one elongated member may be semi-rigid. At least a portion of one constricting surface may be arranged to approximate a contour of the circumscribed tissue. The leakage suppressed may be one of body fluid and air. The device may include a fixation element associated with one constricting surface and arranged to fixate the device to resectioned body tissue. The device may include a fixation element associated with one constricting surface and arranged to engage a receiving element associated with the other elongated constriction surface. The device may also include at least one aperture associated with one constricting surface and arranged for suturing the device to resectioned tissue.

The device may include arrangement of the elements to cooperatively exert a constrictive force of between two and ten pounds per square inch on the circumference of tissue. The constricting surfaces may be moveable together to constrict the circumscribed tissue to an extent necessary to cause the tissue to be excised to become ischemic and necrotic. The first and second constricting surfaces may be arranged for suppressing leakage from resectioned body tissue, including lung tissue, atrial appendage tissue, ovarian tissue, gall bladder tissue, pancreatic tissue, appendix tissue, and spleen tissue. The device may include a medicant that controls biological interaction of the device with the patient. The medicant may be selected from a group consisting of tissue growth inhibitors, tissue growth enhancers, anti-microbial agents such as antibiotic agents or antibacterial agents, anti-inflammatory agents, and biological reaction inhibitors.

The invention further provides an implantable tissue constriction device for suppressing leakage from resectioned body tissue. The device includes a first elongated member having a constricting surface, and a second elongated member having another constricting surface, the constricting surfaces being arranged in combination to circumscribe tissue to be excised, and further being moveable together in opposition to constrict tissue adjacent to the circumscribed tissue to be excised to an extent necessary to suppress leakage from body tissue remaining after excision of the tissue to be excised. The device also includes a bias element coupled to the first member and the second member that brings the constricting surfaces together in opposition. The bias element may be arranged to bring the constricting surfaces together with sufficient force to constrict the circumscribed tissue to an extent necessary to suppress leakage from body tissue remaining after excision of the tissue to be excised. The bias element may be arranged to bring the constricting surfaces together with sufficient force to constrict the circumscribed tissue to an extent necessary to cause the tissue to be excised to become ischemic and necrotic. The bias member may include an elastic material. The device may include a plurality of fixation elements associated with at least one elongated member that grasp resectioned body tissue.

The invention still further provides an implantable device that constrictively suppresses leakage from resectioned body tissue. The device including a first elongated member having a constricting surface, and a second elongated member having another constricting surface. When in receiving configuration, the constricting surfaces are arranged in combination to receive a circumference of tissue adjacent to tissue to be excised, and when in a constricting configuration, the constricting surfaces are arranged to circumscribe and constrict the circumference of tissue sufficiently to suppress leakage from resectioned tissue after excision of the tissue to be excised. The device may include a bias element coupled to the first member and the second member that brings the constricting surfaces together in opposition.

The invention also provides a method of constrictively suppressing leakage from resectioned body tissue. The method includes the steps of placing a first implantable elongated member having a constricting surface arranged to circumscribe a portion of tissue to be excised, and placing a second implantable elongated member having another constricting surface arranged to circumscribe another portion of tissue to be excised, the constricting surfaces being arranged in combination to circumscribe the tissue to be excised. The method also includes the steps of moving the constricting surfaces together in opposition to constrict tissue adjacent to the circumscribed tissue to be excised sufficiently to suppress leakage from the body tissue remaining after excision of the tissue to be excised, and maintaining the constricting surfaces in opposition. The step of moving may include the further step of causing the constricting surfaces to exert a force of between two and ten pounds per square inch on the circumference of tissue. The step of excising the tissue to be excised may include moving the constricting surfaces together sufficiently to isolate the tissue to be excised from fluid communication, and allowing the tissue to be excised to become ischemic and necrotic.

The invention provides yet another implantable tissue constriction device for suppressing leakage from resectioned body tissue. The device includes first elongated means for constricting a portion of a circumference of body tissue adjacent to a tissue to be excised, and second elongated means for constricting the remainder of the circumference of body tissue, the first and second elongated means being moveable together in opposition to constrict tissue adjacent to the circumscribed tissue to be excised to an extent necessary to suppress leakage from body tissue remaining after excision of the tissue to be excised.

The invention still further provides an implantable tissue constriction device for suppressing leakage from a selected body tissue. The device includes first elongated means for constricting a portion of a circumference of tissue adjacent to the selected body tissue, and second elongated means for constricting the remainder of the circumference of tissue, the first and second elongated means being moveable together in opposition to constrict the circumference of tissue to an extent necessary to suppress leakage from the selected body tissue.

The invention additionally provides an implantable tissue constriction device for suppressing leakage from resectioned body tissue. The device includes first elongated means for constricting a portion of a circumference of tissue adjacent to a tissue to be excised, second elongated means for constricting the remainder of the circumference of body tissue, the first and second elongated means being moveable together in opposition to constrict tissue adjacent to the circumscribed tissue to be excised to an extent necessary to suppress leakage from body tissue remaining after excision of the tissue to be excised, and a bias means coupled to the first means and the second means for bringing the constricting surfaces in opposition.

These and various other features as well as advantages which characterize the present invention will be apparent from a reading of the following detailed description and a review of the associated drawings.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a sectional view of a healthy respiratory system;

FIG. 2 illustrates the respiratory system just after suffering an air leak or pneumothorax in a lung lobe;

FIG. 3 illustrates the lobe collapsing and becoming nonfunctional to support respiration;

FIG. 4 is plan view of an implantable tissue constriction device for suppressing leakage from resectioned body tissue, in accordance with an embodiment of the invention;

FIG. 5 is an end view of an implantable tissue constriction device for suppressing leakage from resectioned body tissue, in accordance with an embodiment of the invention;

FIG. 6 illustrates an initial step where the device of FIG. 4 is placed in proximity to a lung lobe, in accordance with an embodiment of the invention;

FIG. 7 illustrates an intermediate step where the device of FIG. 4 is circumscribing a portion of the body tissue adjacent to the tissue to be excised, in accordance with an embodiment of the invention;

FIG. 8 illustrates a final step of device of FIG. 4 suppressing leakage of fluid from a resectioned lung lobe after excision of the tissue to be excised, in accordance with an embodiment of the invention;

FIG. 9 illustrates the device of FIG. 4 implanted within a thorax and suppressing any leakage of fluid from resectioned body tissue, in accordance with an embodiment of the invention;

FIG. 10 is an end view of another implantable tissue constriction device for suppressing leakage from resectioned body tissue, in accordance with an embodiment of the invention;

FIG. 11 is an end view of yet another implantable tissue constriction device for suppressing leakage from resectioned body tissue, in accordance with an embodiment of the invention; and

FIG. 12 is an end view of a folding implantable tissue constriction device for suppressing leakage from resectioned body tissue, in accordance with an embodiment of the invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

Throughout the specification and claims, the following terms take the meanings explicitly associated herein unless the context dictates otherwise. The term “coupled” means either a direct connection between the things that are coupled, or an indirect connection through one or more passive or active intermediary devices. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

While aspects of the invention may be used to suppress leakage from various types of resectioned body tissue, this description will describe an embodiment of the invention being used to suppress leakage from resectioned lung tissue. FIG. 1 is a sectional view of a healthy respiratory system. The respiratory system 20 resides within a thorax 22, which occupies a space defined by a chest wall 24 and a diaphragm 26.

The respiratory system 20 includes the trachea 28, the left mainstem bronchus 30, the right mainstem bronchus 32, and the bronchial branches 34, 36, 38, 40, and 42. The respiratory system 20 further includes left lung lobes 52 and 54 and right lung lobes 56, 58, and 60. Each bronchial branch communicates with a respective different portion of a lung lobe, either the entire lung lobe or a portion thereof.

A healthy respiratory system has an arched or inwardly arcuate diaphragm 26. As the individual inhales, the diaphragm 26 straightens as illustrated in FIG. 1 to increase the volume of the thorax 22. This causes a negative pressure within the thorax. The negative pressure within the thorax in turn causes the lung lobes to fill with air to an inflated condition as illustrated in FIG. 1. When the individual exhales, the diaphragm returns to its original arched condition to decrease the volume of the thorax. The decreased volume of the thorax causes a positive pressure within the thorax which in turn causes exhalation of the lung lobes.

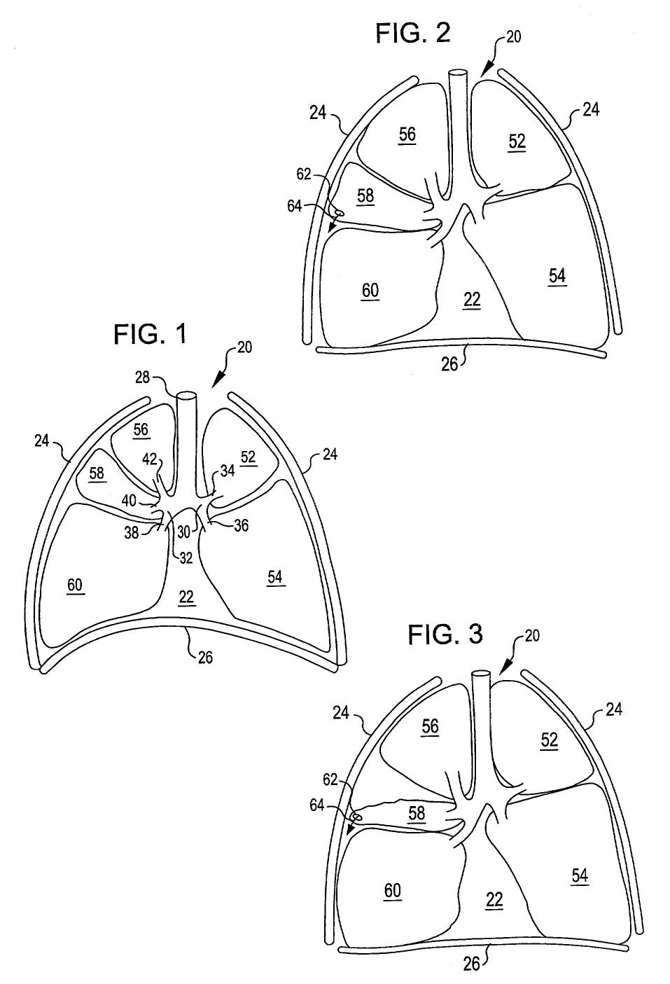

FIG. 2 illustrates the respiratory system 20 just after suffering an air leak or pneumothorax. The air leak or rupture may be from trauma, or from instrumentation such as a surgical procedure excising diseased tissue from the respiratory system. Here it may be seen that the leak 62 has occurred in lung lobe 58. As a result, air is escaping from the lung lobe 58 as indicated by the arrow 64. Hence, this individual is incapable of breathing normally. The negative pressure created by the moving diaphragm 26 causes some of the air taken into lobe 58 to be lost through the rupture 62. When the diaphragm 26 returns to its arched configuration, the positive pressure produced thereby forces still more air from lobe 58 through the rupture. Eventually, within a short time, the lobe 58 collapses as illustrated in FIG. 3 and becomes nonfunctional to support respiration. If the leak 62 suffered by respiratory system 20 had been a blood leak, blood would escape the leak 62 and fill the pleura covering the lung segment. Eventually, the blood leak or hemothorax would interfere with breathing, and also collapse the lobe 58 as illustrated in FIG. 3. Both pneumothorax and hemothorax are life-threatening conditions that must be avoided when performing lung surgery, and remedied when they occur.

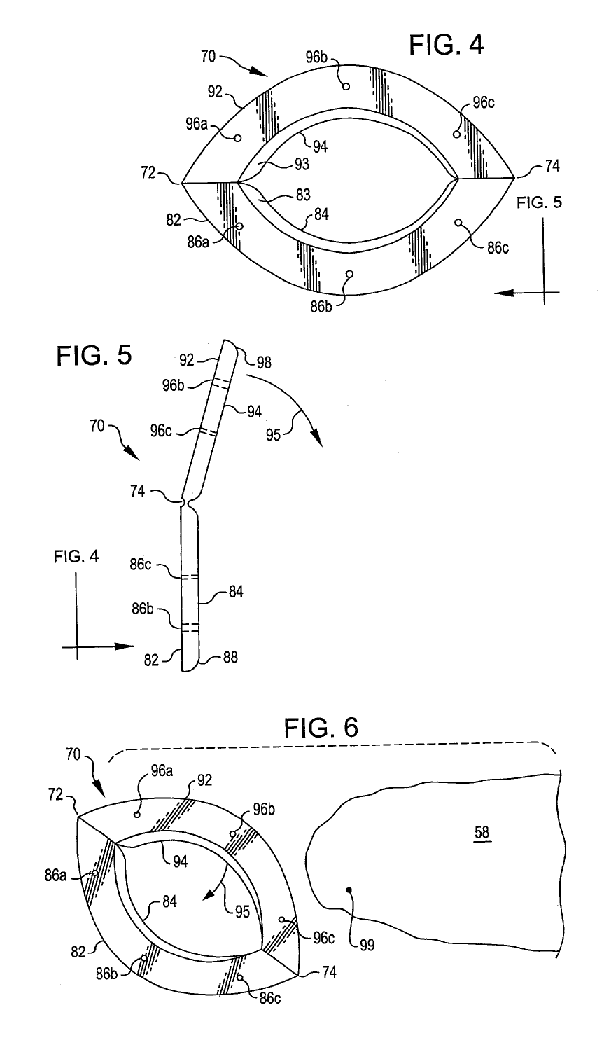

FIGS. 4 and 5 are plan and end views respectively of an implantable tissue constriction device 70 for suppressing leakage from resectioned body tissue, in accordance with an embodiment of the invention. Implantable tissue constriction device 70 includes a first elongated member 82, a second elongated member 92, a first pivot 72, and a second pivot 74. First elongated member 82 includes a first excision guide 83, a first constricting surface 84, a plurality of fixation apertures 86 illustrated as apertures 86a-c, and a radiused edge 88. Second elongated member 92 is similarly arranged with a second excision guide 93, a second constricting surface 94, a plurality of fixation apertures 96 illustrated as apertures 96a-c, and a radiused edge 98.

The first elongated member 82 and second elongated member 92 are made from a semi-rigid biocompatible material suitable for implantation. The degree of semi-rigidity is selected to allow the members 82 and 92 to loosely conform to the periphery of the constricted tissue. Resecting guides 83 and 93 are arranged to guide an excision device, such as a scalpel, in cutting tissue to be excised.

The constricting surfaces 84 and 94 are arranged in combination to circumscribe a portion of the body tissue adjacent to tissue to be excised or sealed from leakage. The radiused edges 88 and 98 are orientated to contact the resectioned body tissue, and are dimensioned to minimize stress that might be imposed on the resectioned body tissue as it transitions into the device 70. The constricting surfaces 84 and 94 are arranged in combination to circumscribe a periphery of tissue adjacent to tissue to be excised. They are further arranged to be moveable together in opposition as indicated by movement arrow 95 to constrict the circumscribed tissue to an extent necessary to suppress leakage from resectioned tissue after excision of the tissue to be excised. In an alternative embodiment, the constricting surfaces 84 and 94 are arranged to be moveable together and to constrict the circumscribed tissue to an extent necessary to cause the tissue to be excised to become ischemic and necrotic.

Pivots 72 and 74 cooperatively allow the constricting surfaces 84 and 94 to open for receiving and circumscribing a portion of body tissue adjacent to the tissue to be excised, and to be moveable and closeable together in opposition as indicated by arrow 95 to constrict body tissue adjacent to the circumscribed tissue portion. The pivots 72 and 74 are illustrated as relieved portions formed in the material of the first and second elongated members 82 and 92. However, in another embodiment, the pivots 72 and 74 are separate elements coupled to the first and second elongated members 82 and 92, such as a flexible or elastic material bridging between the opposed ends of the first and second elongated members 82 and 92. In a further embodiment, the pivots 72 and 74 are mechanical hinge joints that include several parts. The device 70 may include a plurality of fixation apertures 86a-c and 96a-c arranged for suturing the device 70 to the resectioned body tissue. The fixation apertures may be arranged for use with any type of suture material, including elastic suture material.

The physical parameters of the device 70 may be varied according to the tissue type and size to be resectioned, and are selected in part to minimize trauma and stress on the remaining or resectioned tissue. The device 70 may be arranged for suppressing leakage from resectioned body tissue from any organ or tissue, including lung tissue, atrial appendage tissue, ovarian tissue, gall bladder tissue, pancreatic tissue, appendix tissue, and spleen tissue. For example, resectioning lung tissue typically involves a portion of a lung segment that is approximately the size of a fist. The device 70 has a major opening dimension of about five inches and a minor of about two inches for receiving the lung segment. The first and second members 82 and 92 have constricting surfaces approximately 0.5-0.75 inches wide. The first and second members 82 and 92 will have a thickness selected to provide sufficient rigidity to exert a relatively uniform constrictive force against circumscribed tissue and sufficient flexibility to approximate the contour of constricted circumscribed tissue. The device 70 may be made from any biocompatible material suitable for surgical use, such as Teflon and polyurethane.

The elements of the device 70 are further structurally arranged to cooperatively exert a constrictive force of between two and ten pounds per square inch on the circumference of tissue. The device 70 can be arranged to have a low profile for use in relatively small surgical openings, or for use with thorascopic procedures.

FIGS. 6-8 illustrate several steps in suppressing leakage from resectioned tissue employing the implantable tissue constriction device 70, in accordance with an embodiment of the invention. FIG. 6 illustrates an initial step where the device 70 is placed in proximity to the lung lobe 58 and in preparation for excising a tissue to be excised 99. Placement may be through a surgical opening in the chest such as a thoracotomy. The device 70 may have its constricting surfaces 84 and 94 placed adjacent for insertion into the chest, and then moved apart into a receiving configuration in preparation for excising the tissue to be excised 99 as illustrated in FIG. 6. The device 70 may be temporarily carried on a surgical instrument, such as forceps (not shown), for placement in proximity to the lung lobe 58.

Another initial step involves positioning the device 70 with the first constricting surface 84 and the second constricting surface 94 in combination circumscribing tissue of lung lobe 58 adjacent to the tissue to be excised. The circumscribed tissue of lung lobe 58 is selected to minimize the amount of tissue being resected while providing ample opportunity for fixation of the device 70, and is located to constrict the blood supply to the tissue to be excised 99.

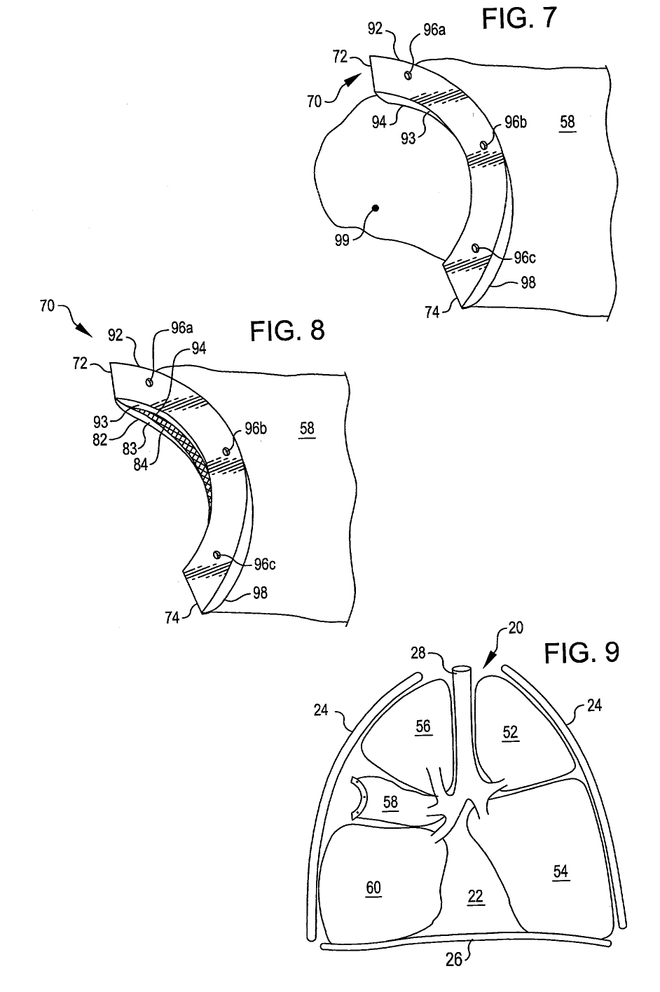

FIG. 7 illustrates an intermediate step where the device 70 is circumscribing a portion of body tissue adjacent to the tissue to be excised 99. The constricting surfaces 84 and 94 have been moved together in opposition to constrict the circumscribed tissue of lung lobe 58 to an extent necessary to suppress leakage from resectioned tissue after excision of the tissue to be excised. The constricting surfaces 84 and 94 may be moved together by squeezing first member 82 and second member 92 together with an instrument, such as forceps or clamps, or an instrument specially arranged for that purpose.

Another intermediate step involves fixating the device 70 to the lung lobe 58 and maintaining constriction on circumscribed tissue. Fixation and maintaining constriction is implemented by suturing the device 70 to the lung lobe 58 using apertures 86 and 96. Any suturing technique and material known in the art may be used. For example, an elastic suture material may be used in a serpentine pattern through apertures 86a-c and 96a-c to both fix device 70 and maintain constriction of the circumscribed tissue. In another embodiment of fixating the device and maintaining constriction, fixation and constriction is initially maintained with a suture through one or more opposed apertures, such as 86a and 96a. Constriction may be augmented with clamps applied to the device 70. After excision, permanent sutures are then placed. While FIG. 7 illustrates the device 70 having one row of three pairs of opposed suture apertures 86a-c and 96a-c, any number of apertures and rows may be used as appropriate to provide a desired level of fixation and compression.

FIG. 8 illustrates a final step of device 70 suppressing leakage of fluid from resectioned lung lobe 58 after excision of the tissue to be excised 99. A final step includes resection of the body tissue illustrated as lung lobe 58 by excising the tissue to be excised 99 and removing it from the body. The tissue is excised using a cutting device, such as a scalpel, which may further include an aid to cutting, such as a thermal heating element, a laser energy emitter, a RF cutting device, or a vibration device. The first excision guide 83 and second excision guide 93 may be used to guide the cutting device. While FIG. 8 illustrates the excision line proximate to guides 83 and 93, the excision may be performed in a manner that leaves a segment of tissue adjacent to guides 83 and 93.

Another final step includes allowing device 70 to remain fixed to the lung portion 58 as an implant and continuously constricting the circumscribed tissue to suppress leakage of fluid. Additional sutures may be placed in apertures 86 and 96 after excision to more permanently fixate device 70 and maintain constriction. The device 70 is expected to provoke a fibrotic response that will aid fixation.

FIG. 9 illustrates the device 70 implanted within the thorax 22 of FIG. 3 and suppressing any leakage of fluid from resectioned body tissue, in accordance with an embodiment of the invention. When the lung lobe 58 is constricted and leakage suppressed as described above, it will thereafter, during successive breaths, re-inflate and become functional once again to support respiration. The use of the device 70 is not restricted to the suppression of air and blood leaks in lungs due to instrumentation and trauma. For example, it may find advantageous use constricting a lung portion suffering from COPD to simulate or achieve lung volume reduction. All of the beneficial effects of lung volume reduction surgery may be realized and, most importantly, without requiring suturing of lung tissue.

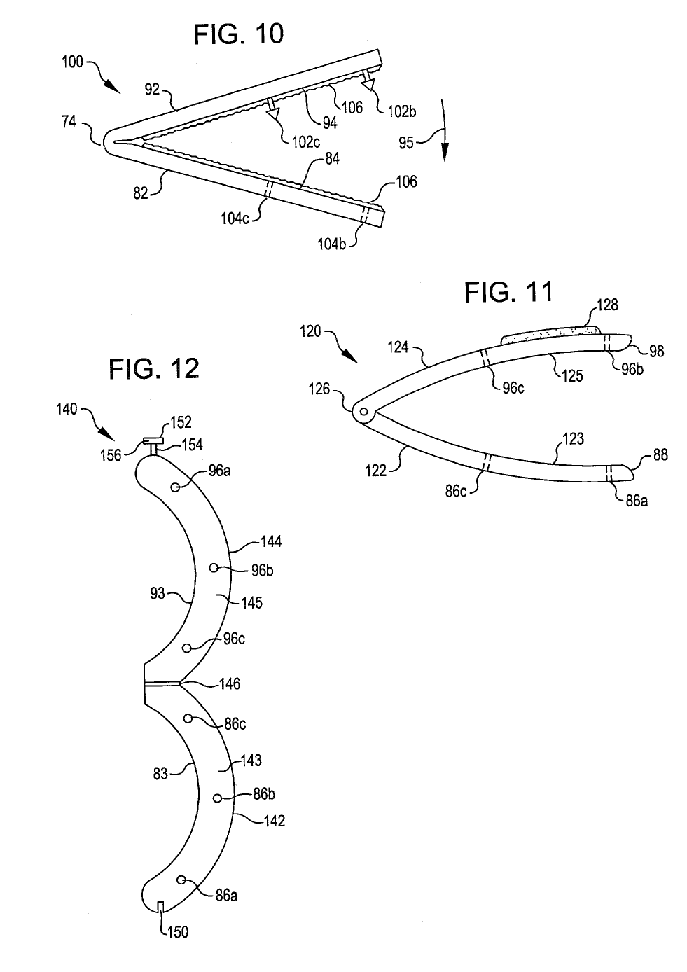

FIG. 10 is an end view of an implantable tissue constriction device 100 for suppressing leakage from resectioned body tissue, in accordance with an embodiment of the invention. Implantable tissue constriction device 100 is similar to the device 70 illustrated in FIGS. 4-5, and provides self-engaging fixation elements and a pliable material. Device 100 includes a first elongated member 82, a second elongated member 92, a first pivot 72 (not shown), and a second pivot 74. First elongated member 82 includes a first constricting surface 84, a plurality of fixation elements 102 illustrated as fixation elements 102b-c, and a pliable material 106. Second elongated member 92 is similarly arranged with a second constricting surface 94, a plurality of fixation element receiving apertures 104 illustrated as receiving apertures 104b-c, and the pliable material 106.

Pliable material 106 is located on at least one constricting surface (84, 94) and is arranged to contact circumscribed tissue and accommodate anatomical variations and irregularities. This accommodation will provide a more uniform distribution of pressure on circumscribed tissue by compensating for variations and irregularities. The accommodation will further reduce trauma and stress on the circumscribed tissue and adjacent tissue. Pliable material 106 may be any pliable material suitable for surgical use, such as silicone.

The fixation elements 102 and opposing receiving apertures 104 are carried on members 82 and 92, respectively, and arranged to fixate device 100 to tissue proximate to, or including, circumscribed tissue, and to maintain constriction on circumscribed tissue. The fixation elements 102 have a tip portion arranged to penetrate the resectioned body tissue, and an engagement portion arranged to engage a corresponding receiving aperture 104. Fixation elements 102 and receiving aperture 104 may be arranged in any manner known in the art for penetrating tissue and engaging each other. In use, device 70 is arranged to be moved as indicated by movement arrow 95 and to constrict circumscribed tissue to an extent necessary to suppress leakage from resectioned tissue after excision of the tissue to be excised in substantially the same manner as device 70. In moving the constricting surfaces 84 and 94 together in opposition, each fixation element pierces through body tissue, moves toward, and engages its corresponding receiving aperture. For example, as the constricting surfaces 84 and 94 are moved together in opposition and to constrict circumscribed tissue, fixation element 102b will pierce through the circumscribed tissue and engage receiving aperture 104b. This provides fixation and maintains constriction for implanting the device 100. The fixation may be augmented by sutures placed in additional fixation apertures that are not shown.

In another embodiment of device 100, fixation elements 102 are arranged to pierce through and engage body tissue, but not engage apertures in device 100. Additional fixation and maintenance of constriction is provided by sutures in a manner similar to that described for device 70.

FIG. 11 is an end view of an implantable tissue constriction device 120 for suppressing leakage from resectioned body tissue, in accordance with an embodiment of the invention. Implantable tissue constriction device 120 is similar in construction and operation to the device 70 illustrated in FIGS. 4-5. Device 120 additionally provides a mechanical hinge 126, a medicant 128, contoured elongated members 122 and 124, and contoured constricting surfaces 123 and 125.

The device 120 requires two mechanical hinges, one of which is illustrated in FIG. 11 as the mechanical hinge 126. Mechanical hinge 126 may be any biocompatible mechanical hinging device suitable for implantation.

Contoured elongated members 122 and 124 and contoured constricting surfaces 123 and 125 are arranged to approximate a contour of tissue circumscribed. The contour will depend on the kind of tissue or organ being circumscribed, and may be created in members 122 and 124 by curving them as illustrated in FIG. 11. In an alternative embodiment, the contour is created by using planar members and shaping contoured constricting surfaces 123 and 125.

While illustrated as carried on elongated member 124, medicant 128 may be associated with any portion of device 120. The medicant 128 is provided to control biological interaction of the device 120 with the patient. For example, an anti-microbial agent may be selected to control infection, or a tissue growth enhancer may be selected to encourage growth of fibrotic tissue to assist in fixating device 120 to the lung lobe 58. The medicant 128 can be any medicant suitable for association with an implanted device, and may include tissue growth inhibitors, tissue growth enhancers, anti-microbial agents such as antibiotic agents or antibacterial agents, anti-inflammatory agents, and biological reaction inhibitors.

FIG. 12 is an end view of a folding implantable tissue constriction device 140 for suppressing leakage from resectioned body tissue, in accordance with an embodiment of the invention. Folding device 140 is similar in construction and operation to device 70, except it is designed to fold around a single pivot 146 and maintain constriction with a bias element 152.

Folding device 140 includes a first elongated member 142, a second elongated member 144, a pivot 146, and a bias element 152. The first and second elongated members 142 and 144 are similar to the device 70, and additionally provide a bias element 152 and a single pivot 146. First elongated member 142 includes a constricting surface 143, a plurality of fixation apertures 86 illustrated as apertures 86a-c, a first excision guide 83, and a bias element engaging aperture 150. Second elongated member 144 is similarly arranged with a second excision guide 93, a second constricting surface 145, a plurality of fixation apertures 96 illustrated as apertures 96a-c, and the bias element 152.

The first elongated member 142 and the second elongated member 144 are rotatably coupled by pivot 146, making the constricting surfaces 143 and 145 moveable together in opposition about pivot 146 to circumscribe a portion of body tissue adjacent to the tissue to be excised 99. Pivot 146 is arranged to allow device 140 to lay flat in substantially a single plane when in an open configuration. Pivot 146 is also arranged to allow the first and second members 142 and 144 to pivotally move from the open configuration to a constricting configuration, much like closing an open book. Any type of pivot known in the art may be used. The pivot 146 may be relieved portions formed in the material of the first and second elongated members 142 and 144 in a manner similar to that described for device 70 in FIGS. 4-5; it may be a separate element coupled between ends of the first and second elongated members 142 and 146, such as a flexible or elastic material bridging between ends; or a mechanical hinge joint formed in a manner similar to that described for hinged device 120 in FIG.

The bias element 152 is arranged to engage the bias element engaging aperture 150. Bias element 152 includes a bias portion 154 and an engaging portion 156. When engaging portion 156 is engaged in the bias element engaging aperture 150, bias element 152 is arranged to bring the constricting surfaces 143 and 145 together. Bias element 152 is arranged either alone, or in cooperation with sutures placed in fixation apertures 86 and 96, to provide sufficient force to constrict circumscribed tissue to an extent necessary to suppress leakage from resectioned tissue after excision of the tissue to be excised 99. In another embodiment, the bias element 152 is arranged either alone, or in cooperation with sutures placed in fixation apertures 86 and 96, to provide sufficient force to bring the constricting surfaces 143 and 145 together with sufficient force to constrict the circumscribed tissue to an extent necessary to cause the tissue to be excised 99 to become ischemic and necrotic. The bias portion 154 is made from a biocompatible elastic material having elasticity sufficient to provide the necessary constriction, and may include a silicone material (polydimethelsilaxone), a polyurethane, or a polypropylene. Engaging portion 152 and bias element engaging aperture 150 may be any arrangement of materials and shapes that provide engagement and retention.

In operation, implanting the folding device 140 to constrictively suppress leakage from resectioned body tissue is performed using steps similar to implanting device 70. However, the device 140 may have an advantage over device 70 in certain situations because it lies substantially in a single plane when in an open configuration, requiring a smaller chest opening for placement in proximity to lung lobe 58. An initial step involves positioning the folding device 140 in proximity to tissue to be circumscribed, and folding the device 140 about its pivot 146 to move the first constricting surface 143 and the second constricting surface 145 in combination to circumscribe tissue of lung lobe 58 adjacent to tissue to be excised 99. As described with respect to device 70, the constricting surfaces 143 and 145 may be moved together by squeezing first member 142 and second member 144 together with an instrument, such as forceps or clamps, or an instrument specially arranged for that purpose. Folding device 140 provides for the constricting surfaces 143 and 145 to be further moved together and constriction maintained by stretching the bias portion 154 and engaging it in engaging portion 156, and releasing bias portion 154 to bring the constricting surfaces 143 and 145 together. Bias portion 154 is stretched and engaged into portion 156 by gripping engaging portion 156 with an instrument such as a clamp or forceps, or fingers.

Although the present invention has been described in considerable detail with reference to certain preferred embodiments, other embodiments are possible. Therefore, the spirit or scope of the appended claims should not be limited to the description of the embodiments contained herein. It is intended that the invention resides in the claims hereinafter appended.

What is claimed is:

1. An implantable tissue constriction device for suppressing leakage from body tissue, the device comprising:
a first elongated member having a constricting surface; and
a second elongated member having another constricting surface, the constricting surfaces being arranged in combination to circumscribe a portion of the body tissue, and further being moveable together in opposition to constrict body tissue adjacent to the circumscribed body tissue portion to an extent necessary to suppress leakage from the body tissue.

2. An implantable tissue constriction device for suppressing leakage from resectioned body tissue, the device comprising:
a first elongated member having a constricting surface; and
a second elongated member having another constricting surface, the constricting surfaces being arranged in combination to circumscribe tissue to be excised, and further being moveable together in opposition to constrict tissue adjacent to the circumscribed tissue to be excised to an extent necessary to suppress leakage from body tissue remaining after excision of the tissue to be excised.

3. The device of claim 2, wherein at least one elongated constrictive surface includes a pliable material arranged to contact the circumscribed tissue and accommodate anatomical variations.

4. The device of claim 2, wherein the device further includes a pivot coupled between the first elongated member and the second elongated member, about which the constricting surfaces are moveable together in opposition.

5. The device of claim 2, wherein the device further includes a first pivot and a second pivot, each pivot coupling the first constrictive surface to the second constrictive surface, and cooperatively allowing movement of the constricting surfaces together in opposition.

6. The device of claim 2, wherein at least one elongated member is semi-rigid.

7. The device of claim 2, wherein the leakage suppressed is one of body fluid and air.

8. The device of claim 2, wherein at least a portion of one constricting surface is arranged to approximate a contour of the circumscribed tissue.

9. The device of claim 2, further including a fixation element associated with one constricting surface and arranged to fixate the device to resectioned body tissue.

10. The device of claim 2, further including a fixation element associated with one constricting surface and arranged to engage a receiving element associated with the other elongated constriction surface.

11. The device of claim 2, further including at least one aperture associated with one constricting surface and arranged for suturing the device to resectioned tissue.

12. The device of claim 2, further including the elements of the device being arranged to cooperatively exert a constrictive force of between two and ten pounds per square inch on the circumference of tissue.

13. The device of claim 2, wherein the constricting surfaces are further moveable together to constrict the circumscribed tissue to an extent necessary to cause the tissue to be excised to become ischemic and necrotic.

14. The device of claim 2, wherein the first and second constricting surfaces are arranged for suppressing leakage from resectioned body tissue of one of a lung tissue, atrial appendage tissue, ovarian tissue, gall bladder tissue, pancreatic tissue, appendix tissue, and spleen tissue.

15. The device of claim 2, further comprising a medicant that controls biological interaction of the device with the patient.

16. The device of claim 15, wherein the medicant is selected from a group consisting of tissue growth inhibitors, tissue growth enhancers, anti-microbial agents such as antibiotic agents or antibacterial agents, anti-inflammatory agents, and biological reaction inhibitors.

17. An implantable tissue constriction device for suppressing leakage from resectioned body tissue, the device comprising:
a first elongated member having a constricting surface;
a second elongated member having another constricting surface, the constricting surfaces being arranged in combination to circumscribe tissue to be excised, and further being moveable together in opposition to constrict tissue adjacent to the circumscribed tissue to be excised to an extent necessary to suppress leakage from body tissue remaining after excision of the tissue to be excised; and
a bias element coupled between the first member and the second member that brings their constricting surfaces together in opposition.

18. The device of claim 17, wherein the bias element is arranged to bring the constricting surfaces together with sufficient force to constrict the circumscribed tissue to an extent necessary to suppress leakage from resectioned tissue after excision of the tissue to be excised.

19. The device of claim 17, wherein the bias element is arranged to bring the constricting surfaces together with sufficient force to constrict the circumscribed tissue to an extent necessary to cause the tissue to be excised to become ischemic and necrotic.

20. The device of claim 17, wherein the bias member includes an elastic material.

21. The device of claim 17, further including a plurality of fixation elements associated with at least one elongated member that grasp resectioned body tissue.

22. An implantable device that constrictively suppresses leakage from resectioned body tissue, the device comprising:
a first elongated member having a constricting surface; and
a second elongated member having another constricting surface, when in a receiving configuration the constricting surfaces being arranged in combination to receive a circumference of tissue adjacent to tissue to be excised, and when in a constricting configuration the constricting surfaces being arranged to circumscribe and constrict the circumference of tissue sufficiently to suppress leakage from body tissue remaining after excision of the tissue to be excised.

23. The device of claim 22, further including a bias element coupled to the first member and the second member that brings the constricting surfaces together in opposition.

24. A method of constrictively suppressing leakage from resectioned body tissue, the method including the steps of:
placing a first implantable elongated member having a constricting surface arranged to circumscribe a portion of tissue to be excised;
placing a second implantable elongated member having another constricting surface arranged to circumscribe another portion of tissue to be excised, the constricting surfaces being arranged in combination to circumscribe the tissue to be excised;
moving the constricting surfaces together in opposition to constrict tissue adjacent to the circumscribed tissue to be excised sufficiently to suppress leakage from the body tissue remaining after excision of the tissue to be excised; and
maintaining the constricting surfaces in opposition.

25. The method of claim 24, wherein the step of moving includes the further step of causing the constricting surfaces to exert a force of between two and ten pounds per square inch on the circumference of tissue.

26. The method of claim 24, further including the step of excising the tissue to be excised by moving the constricting surfaces together sufficiently to isolate the tissue to be excised from fluid communication, and allowing the tissue to be excised to become ischemic and necrotic.

27. An implantable tissue constriction device for suppressing leakage from resectioned body tissue, the device comprising:
first elongated means for constricting a portion of a circumference of body tissue adjacent to a tissue to be excised; and
second elongated means for constricting the remainder of the circumference of body tissue, the first and second elongated means being moveable together in opposition to constrict tissue adjacent to the circumscribed tissue to be excised to an extent necessary to suppress leakage from body tissue remaining after excision of the tissue to be excised.

28. An implantable tissue constriction device for suppressing leakage from a selected body tissue, the device comprising:
first elongated means for constricting a portion of a circumference of tissue adjacent to the selected body tissue; and
second elongated means for constricting the remainder of the circumference of tissue, the first and second elongated means being moveable together in opposition to constrict the circumference of tissue to an extent necessary to suppress leakage from the selected body tissue.

29. An implantable tissue constriction device for suppressing leakage from resectioned body tissue, the device comprising:
first elongated means for constricting a portion of a circumference of tissue adjacent to a tissue to be excised;
second elongated means for constricting the remainder of the circumference of body tissue, the first and second elongated means being moveable together in opposition to constrict tissue adjacent to the circumscribed tissue to be excised to an extent necessary to suppress leakage from body tissue remaining after excision of the tissue to be excised; and
a bias means coupled to the first means and the second means for bringing the constricting surfaces in opposition.
